(19) United States
(12) Patent Application Publication
(10) Pub. No.: US 2025/0147030 A1
(43) Pub. Date: May 8, 2025

(54) PEPTIDE-ENCODED LIBRARIES OF SMALL MOLECULES FOR DE NOVO DRUG DISCOVERY

(71) Applicant: Massachusetts Institute of Technology, Cambridge, MA (US)

(72) Inventors: Stephen L. Buchwald, Newton, MA (US); Bradley L. Pentelute, Revere, MA (US); Nathalie Grob, Cambridge, MA (US); Simon Roessler, Cambridge, MA (US)

(21) Appl. No.: 18/712,113

(22) PCT Filed: Dec. 5, 2022
(86) PCT No.: PCT/US22/51802

§ 371 Date: May 21, 2024

Related U.S. Application Data
(60) Provisional application No. 63/285,693, filed on Dec. 3, 2021.

(51) Int. Cl. G01N 33/58; G01N 33/68

(52) U.S. Cl. CPC G01N 33/58; G01N 33/6824; G01N 33/6848

(57) ABSTRACT

Disclosed are conjugates and methods of identifying ligands for substrates using said conjugates. Also disclosed herein are methods of making the conjugates.

### RELATED APPLICATIONS

This application claims the benefit of priority to U.S. Provisional Patent Application No. 63/285,693, filed on Dec. 3, 2021, the contents of which are hereby incorporated by reference in their entirety.

### BACKGROUND

Identification of chemical compounds that bind to targets of interest represents a fundamental step in de novo drug discovery and development of chemical probes. This endeavor relies on the efficient evaluation of a vast amount of chemically diverse potential binders. Combinatorial libraries are collections of chemical compounds synthesized by reactions of multiple different combinations of related chemical species. The resulting compounds can be elaborated further in a similar combinatorial fashion to afford exponentially large mixtures of compounds. Such libraries offer a valuable source of binders which can be identified through a process termed affinity selection (AS), wherein an immobilized target of interest is incubated with the library allowing for subsequent enrichment of binders by removal of all library members lacking affinity through washing. This process enables the simultaneous evaluation of a vast amount of potential binders and has emerged as invaluable part of de novo drug discovery and development of chemical probes. The key difficulty arises after AS, namely the identification of retained binders. A powerful approach for this analytic challenge involves the inclusion of a covalently bound encoding tag for each library member, installed through parallel elaboration of a bifunctional chemical compound during combinatorial library synthesis.

The most commonly used encoding strategy relies on DNA encoding tags, which enable readout through highly sensitive PCR amplification and analysis. After three decades of development, DNA-encoded libraries (DELs) have become a staple in the toolbox of drug discovery and have enabled the discovery of a manifold of new binders for therapeutic targets in both academic and industrial research programs. Despite their great potential, DNA-based encoding tags suffer from several drawbacks. The preparation of chemically diverse DELs is limited by synthetic methods compatible with DNA, which necessitates aqueous solvents and can undergo loss of encoding information through depurination in the presence of metals or strong acids. This challenge was already recognized in the initial report on DELs by Brenner and Lerner, and numerous contrivances have since been developed in order to remedy some of the synthetic limitations. Nonetheless, a recent reaction rehearsal by Paegel found that many valuable synthetic manipulations used in the preparation of DELs leave only a fraction of encoding information viable. A further limitation of DELs arises in regards to potential targets. The presence of DNA inherently precludes the efficient selection of potential DNA-chelating molecules as well as the use of proteins of interest interacting with DNA, which includes therapeutically relevant targets such as transcription factors. Given the great potential of encoded small molecule libraries, a novel encoding approach which could address the shortcomings of DNA is highly desirable.

### SUMMARY OF THE INVENTION

In one aspect, the present disclosure provides conjugates having a structure represented by formula I or a salt thereof:

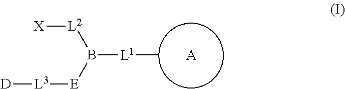

wherein
A is a resin;
B is a branching unit;
D is hydrogen or a peptide;
E is a cleavable moiety;
X is hydrogen, halo, amino, carboxy, protected, amino, protected carboxy, a small molecule, alkyl, alkene, alkyne, aryl, ketone, acyl, aldehyde, hydroxy, or a plurality of small molecules;
L¹ is a cleavable covalent linker;
L² is a covalent linker; and
L³ is a covalent linker.

In another aspect, the present disclosure provides methods of identifying a ligand for a substrate, comprising contacting the substrate with the conjugate disclosed herein, wherein X is a small molecule or a plurality of small molecules; and D is an encoding peptide tag.

In another aspect, the present disclosure provides kits for performing the methods disclosed herein, wherein the kit comprises a conjugate disclosed herein, D is hydrogen, amino, protected amino, carboxy, or protected carboxy; and X is halo, amino, protected amino, or protected carboxy.

In another aspect, the present disclosure provides methods of making a conjugate disclosed herein, wherein X is a small molecule or a plurality of small molecules; and D is an encoding peptide tag; comprising:
i) providing a conjugate disclosed herein, wherein X is halo, protected amino, or protected carboxy, and D is hydrogen;
ii) contacting the conjugate with an encoding amino acid disclosed herein or a spacing monomer of disclosed herein and a peptide coupling reagent;
iii) optionally repeating step ii);
iv) protecting the N-terminus or C-terminus of the encoding peptide tag;
v) deprotecting X;
vi) contacting the conjugate with:
   a) a small molecule comprising a carboxy moiety or an amino moiety and a peptide coupling reagent; or
   b) a small molecule comprising a halo moiety and a catalyst for performing a metal mediated cross-coupling reaction;
vii) optionally repeating step vi); and
viii) deprotecting the N-terminus or C-terminus of the encoding peptide tag, thereby providing a conjugate, wherein X is a small molecule or a plurality of small molecules, and D is an encoding peptide tag.

In another aspect, the present disclosure provides methods of making a conjugate disclosed herein, wherein X is a small molecule or a plurality of small molecules, and D is an encoding peptide tag; comprising:
I) providing a conjugate disclosed herein, wherein X is halo, amino, or carboxy, and D is a protected carboxy or protected amino;
II) contacting the conjugate with:
   a) a small molecule comprising a carboxy moiety or an amino moiety and a peptide coupling reagent; or
   b) a small molecule comprising a halo moiety and a catalyst for performing a metal mediated cross-coupling reaction;
III) optionally repeating step II);
IV) deprotecting D;
V) contacting the conjugate with an encoding amino acid of disclosed herein or a spacing monomer of disclosed herein and a peptide coupling reagent, thereby yielding a conjugate, wherein X is a small molecule or a plurality of small molecules; and D is an encoding peptide tag; and
VI) optionally repeating step V).

### BRIEF DESCRIPTION OF THE DRAWINGS

FIG. 1 shows a schematic for the development of Peptide-Encoded Small Molecule Libraries (PELs). The development of PELs hinges on the implementation of an efficient synthetic framework as well as enrichment and decoding protocols.

FIG. 2 shows the design and elaboration of the synthetic framework. Orthogonal protecting groups allow for parallel synthetic elaboration while orthogonally cleavable linkers enable the isolation of the library after synthesis and the liberation of the peptide tag after AS before sequencing.

FIG. 3 shows exemplary 16 encoding monomers with unique exact masses given in brackets.

FIG. 4 shows an exemplary encoding tag.

FIG. 5 shows the design and workflow of an exemplary tag. BRD4(1) ligand (+)-JQ1 was coupled to a model peptide tag according to the library design. Folic acid was coupled to a model peptide tag according to the library design. MagBead enrichment with the folic acid model conjugate and FOLR1 was then carried out.

FIGS. 6A-6F show the results of the enrichment of a model tag. EIC (488.95-488.99, corresponds to m/z of [model tag+3H]³⁺) after incubation of streptavidin (6A) or FOLR1 (6B) with 500 pM folic acid model conjugate (retention time=33.5-34.0 min), and average local confidence scores from de novo sequencing (6C). EIC after incubation of streptavidin (6D) or BRD4(1) (6E) with 1000 pM JQ-1 model conjugate (retention time=33.5-34.0 min), and average local confidence scores from de novo sequencing (6E).

FIG. 7 shows exemplary palladium-mediated C—N cross-coupling reactions that can be used to attach small molecules to PELs. All reactions were conducted at 5 μmol scale in sealed HPLC vials agitated at 200 rpm in a heated incubator (50° C.). Percentage given represents LC-MS TIC integration of the product peak against all other peptidic peaks not present in the starting material.

FIG. 8 shows exemplary palladium-mediated Suzuki-Miyaura coupling reactions that can be used to attach small molecules to PELs. All reactions were conducted at 5 μmol scale in sealed microcentrifuge tubes agitated on a nutating mixer. Percentage given represents LC-MS TIC integration of the product peak against all other peptidic peaks not present in the starting material.

FIG. 9 shows a carboxylic acid coupling that can be used to attach small molecules to PELs and associated yield histogram. All reactions were conducted at 3 μmol scale in fritted syringes, yield distribution shown as histogram with a vertical dotted line marking a 70% threshold.

FIG. 10 shows the exemplary trifunctional building block validation. Reactions were conducted on a 10 μmol scale.

FIG. 11 shows the exemplary synthesis of two PELs.

FIG. 12 shows two model library compounds and the corresponding LC-MS. Synthesis of two model compounds using the same procedure as during combinatorial library synthesis affords products after 40 steps.

### DETAILED DESCRIPTION OF THE INVENTION

De novo sequencing of large peptides libraries through nLC-MS/MS has previously been disclosed. These findings led us to hypothesize that a small molecule could in theory be encoded by a specific peptide sequence. Such a peptide-encoded library (PEL) would require a multidimensional optimization effort including the development of a suitable framework featuring orthogonal protecting groups and linkers, the implementation of synthetic chemistry suitable for PELs, and an amino acid set optimized for encoding, enrichment, and sequencing.

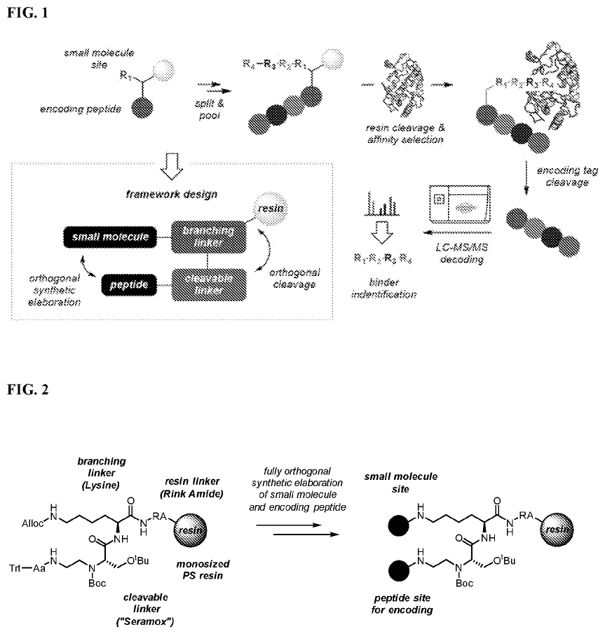

The ability to efficiently sequence peptides enriched through AS from complex mixtures offers the opportunity to develop a small molecule combinatorial library platform featuring peptides as encoding tags. This endeavor necessitates the solution of a complex multifaceted problem which includes development of a suitable synthetic framework allowing the modular preparation of small molecule peptide conjugates, optimization of an efficient AS enrichment protocol, identification of peptide monomers and mass spectrometry methods which allow highly efficient de novo sequencing, and implementation of efficient small molecule chemistry compatible with the peptide conjugate (FIG. 1).

The efforts towards the realization of such a complex system commences with the development of the synthetic framework, which requires orthogonality in the parallel solid phase synthetic elaboration of the small molecule and peptidic encoding site as well as cleavage of the library after synthesis and of the tag after AS before sequencing. Efficiency and selectivity in these orthogonal functional handles are crucial for successful implementation of PELs.

An established synthetic framework allows the exploration of parameters involved in affinity enrichment and sequencing, such as an optimized set of amino acids and tag structure, as well as selection and nLC-MS/MS parameters. Particular challenges are the identification of encoding peptides, which exhibit resilience to a variety of synthetic conditions and allow for high-confidence de novo sequencing. The potential hydrophobicity of the resulting conjugates could hamper the selection efficiency through limited solubility in aqueous incubation media and non-specific binding. The encoding tag can be designed to include basic peptides known to enhance sequencing at specific sites in polypeptides as well as increase hydrophilicity. With the well-defined tag structures in hand, a thorough optimization of nLC-MS/MS methods allows the implementation of a confident and sensitive decoding of tags.

The success of PELs hinges on the adaption of valuable synthetic methods currently used in drug discovery for the preparation of structurally diverse compounds. The synthesis is implemented on solid support, thus enabling efficient functionalization through large excess of reagents which can be readily washed away upon completion of the reaction. Amide bond formation represents a fundamental reaction in solid-phase peptide synthesis (SPPS) and is expected to be readily utilized in the diversification of appropriate small molecule coupling partners. Analysis of the most frequently used chemical reactions in medicinal chemistry found amide bond formation as the most used reaction. The same analysis revealed that only two of the most used reactions were discovered in the last four decades, namely the Suzuki-Miyaura and Buchwald-Hartwig reaction. Both of these reactions represent staples in the medicinal chemists' toolbox for the preparation of therapeutically relevant target molecules, but have found limited success in DELs in part due to the presence of metal ions which may induce loss of encoding information. Efficient protocols for the palladium-mediated formation of C—C and C—N bonds compatible with solid-phase synthesis and encoding peptide tags allows for preparation of chemically diverse PELs with high purity. Additionally, a wide array of diversity-oriented reactions featuring DNA-incompatible conditions can be explored. Any method adapted or developed for this system must allow for implementation in parallel combinatorial synthesis in order to allow for library synthesis.

In one aspect, the present disclosure provides conjugates having a structure represented by formula I or a salt thereof:

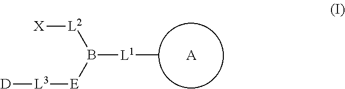

wherein
A is a resin;
B is a branching unit;
D is hydrogen, a carboxy protecting group, an amino protecting group, or an encoding peptide tag;
E is a cleavable moiety;
X is hydrogen, halo, amino, carboxy, protected, amino, protected carboxy, a small molecule, alkyl, alkene, alkyne, aryl, ketone, acyl, aldehyde, hydroxy, or a plurality of small molecules;
L¹ is a cleavable covalent linker;
L² is a covalent linker; and
L³ is a covalent linker.

In certain embodiments, the resin is a polymer resin. In certain embodiments, the resin is a polystyrene resin or a polystyrene co-polymer resin.

In certain embodiments, L¹ is alkylenyl, alkenylenyl, or alkynylenyl. In certain embodiments, L¹ is substituted with amide or carboxy. In certain embodiments, L¹ is Rink Amide, Wang amide, HMPA, Rink acid, HMPB, trityl and derivatives, SASRIN, HAL, PAL, Sieber amide, HMBA, 3-nitro-4-methoxylmethyl benzoyl, 3-nitro-4-aminomethyl benzoyl, alpha-methyl-6-nitro-veratrylamine based handles, or any other orthogonally cleavable linker. In certain embodiments, L¹ is Rink Amide.

In certain embodiments, B is a trifunctional moiety. In certain embodiments, B is a nitrogen atom or a carbon atom. In certain embodiments, B is an amide. In certain embodiments, B is

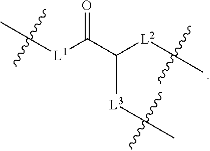

In certain embodiments, L² is alkyl, alkylenyl, alkenylenyl, or alkynylenyl. In certain embodiments, L² is alkylenyl. In certain embodiments, L² is heteroalkylenyl. In certain embodiments, L² is alkyl. In certain embodiments, L² is arakylamidoalkyl, arakylacylalkyl, arakylcarbamatealkyl, arakylarylalkyl arakylheteroarylalkyl, arakylsulfonamidealkyl, or arakylureaalkyl. In certain embodiments, L² is heteroarakylamidoalkyl, heteroarakylacylalkyl, heteroarakylcarbamatealkyl, heteroarakylarylalkyl heteroarakylheteroarylalkyl, heteroarakylsulfonamidealkyl, or heteroarakylureaalkyl. In certain embodiments, L² is heterocyclylamidoalkyl, heterocyclylacylalkyl, heterocyclylcarbamatealkyl, heterocyclylarylalkyl heterocyclylheteroarylalkyl, heterocyclylsulfonamidealkyl, or heterocyclylureaalkyl. In certain embodiments, L² is arakylamidoalkyl or heterocyclylamidoalkyl.

In certain embodiments, X is hydrogen. In certain embodiments, X is halo (e.g., bromo). In certain embodiments, X is amino (e.g., NH₂ or NH). In certain embodiments, X is protected amino. In certain embodiments, X is carboxy or protected carboxy. In certain embodiments, X is a small molecule. In certain embodiments, X is a plurality of small molecules. In certain embodiments, the small molecule is a drug. In certain embodiments, each small molecule is a drug.

In certain embodiments, E cleaved by an acid, base, light, heat, or an enzyme. In certain embodiments, E is heteroalkylenyl (e.g., aminoalkylenyl or carboxyalkylenyl). In certain embodiments, E is an amino acid. In certain embodiments, E is:

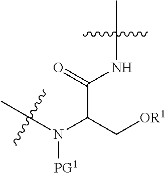

wherein,
R¹ is alkyl; and
PG¹ is a nitrogen protecting group.

In certain embodiments, the nitrogen protecting group is tert-butyloxycarbonyl. In certain embodiments, L³ is a bond, alkyl, alkylenyl, alkenylenyl, or alkynylenyl. In certain embodiments, L³ is alkylenyl. In certain embodiments, L³ is heteroalkylenyl. In certain embodiments, L³ is aminoalkyl (e.g., aminoethyl). In certain embodiments, L³ is a bond.

In certain embodiments, D is hydrogen. In certain embodiments, D is amino. In certain embodiments, D is protected amino. In certain embodiments, D is carboxy. In certain embodiments, D is protected carboxy. In certain embodiments, D is an encoding peptide tag.

In certain embodiments, the encoding peptide tag comprises plurality of encoding amino acids. In certain embodiments, the encoding peptide tag comprises 2-50 encoding amino acids. In certain embodiments, the encoding peptide tag comprises 2, 3, 4, 5, 6, 7, 8, 9, 10, 11, 12, 13, 14, 15, 16, 17, 18, 19, or 20 encoding amino acids.

In certain embodiments, the encoding amino acids are naturally occurring amino acids. In certain embodiments, the encoding amino acids are D-enantiomers of naturally occurring amino acids. In certain embodiments, the encoding amino acids are non-isobaric non-canonical amino acids. In certain embodiments, the encoding amino acids are each independently selected from the group consisting of Ala, Abu, Ser, Pro, Val, Thr, Cpa, Hyp, Leu, Mox, Cba, Aoa, Phe, Cha, Tyr, and Dmf. In certain embodiments, at least one of the encoding amino acids are in the L configuration. In certain embodiments, at least one of the encoding amino acids are in the D configuration.

In certain embodiments, the encoding peptide tag further comprises one or more spacing monomer(s). In certain embodiments, the encoding peptide tag further comprises 1, 2, 3, 4, 5, 6, 7, 8, 9, 10, 11, 12, 13, 14, 15, 16, 17, 18, 19, or 20 spacing monomer(s). In certain embodiments, the encoding peptide tag terminates in a C-terminus. In certain embodiments, the encoding peptide tag comprises a spacing monomer or an oxygen protecting group at the C-terminus of the peptide. In certain embodiments, the encoding peptide tag comprises a spacing monomer or an oxygen protecting group at the C-terminus of the peptide. In certain embodiments, the encoding peptide tag comprises an oxygen protecting group at the C-terminus of the peptide. In certain embodiments, the oxygen protecting group is a trityl. In certain embodiments, the encoding peptide tag terminates in an N-terminus. In certain embodiments, the encoding peptide tag comprises a spacing monomer or an oxygen protecting group at the N-terminus of the peptide. In certain embodiments, the encoding peptide tag comprises a spacing monomer at the N-terminus of the peptide. In certain embodiments, the encoding peptide tag comprises a nitrogen protecting group at the N-terminus of the peptide. In certain embodiments, the nitrogen protecting group is a trityl, Alloc, Dde, or ivDde. In certain embodiments, the nitrogen protecting group is a trityl.

In certain embodiments, the encoding peptide tag comprises a spacing monomer after every first, second, third, or fourth encoding amino acid. In certain embodiments, the encoding peptide tag comprises a spacing monomer after every second amino acid. In certain embodiments, at least one spacing monomer is apolar. In certain embodiments, at least one spacing monomer is basic. In certain embodiments, each spacing monomer is independently selected from naturally occurring amino acids. In certain embodiments, each spacing monomer is independently selected from the group consisting of Lys, Arg, homo-Arg, ornithine, and diaminobutyric acid.

In another aspect, the present disclosure provides methods of identifying a ligand for a substrate comprising contacting the substrate with the conjugate disclosed herein, wherein X is a small molecule or a plurality of small molecules; and D is an encoding peptide tag.

In certain embodiments, the substrate is a protein (e.g., an enzyme). In certain embodiments, the substrate is a polynucleotide (e.g., DNA or RNA).

In certain embodiments, the methods further comprise incubating the substrate with the conjugate. In certain embodiments, the methods further comprise cleaving a bond between L¹ and A. In certain embodiments, the methods further comprise cleaving a bond between B and L¹. In certain embodiments, the methods further comprise denaturing the substrate.

In certain embodiments, the method further comprises a step of deconvoluting the ligand. In certain embodiments, the step of deconvoluting the ligand comprises:
cleaving L³, thereby yielding a free peptide tag;
optionally isolating the free peptide tag;
optionally purifying the free peptide tag; and
determining the molecular weight or retention time of the free peptide tag.

In certain embodiments, L³ is cleaved via oxidative cleavage, acidic cleavage, or basic cleavage. In certain embodiments, L³ is cleaved via oxidative cleavage.

In certain embodiments, the free peptide tag is isolated. In certain embodiments, the free peptide tag is isolated by high performance liquid chromatography. In certain embodiments, the molecular weight of free peptide tag is determined. In certain embodiments, the molecular weight and the sequence of the free peptide tag is determined by mass spectrometry. In certain embodiments, the molecular weight and the sequence of the free peptide tag is determined by the analysis of mass fragmentation patterns. In certain embodiments, the retention time of free peptide tag is determined. In certain embodiments, the retention time of free peptide tag is determined via liquid chromatography. In certain embodiments, the molecular weight, sequence, and/or retention time of free peptide tag are concurrently determined by LC-MS or LC-MS/MS. In certain embodiments, the sequence of the free peptide tag is determined. In certain embodiments, the sequence of the free peptide tag is determined by Edman degradation.

In certain embodiments, identifying the ligand comprises comparing the molecular weight or retention time of the free peptide tag to a database or list comprising the identity of the small molecule or the plurality of small molecules.

In another aspect, the present disclosure provides kits for performing the methods disclosed herein, wherein the kit comprises a conjugate disclosed herein, wherein D is hydrogen, amino, protected amino, carboxy, or protected carboxy; and X is hydrogen, halo, amino, protected amino, or protected carboxy.

In certain embodiments, the kit further comprises a peptide coupling reagent or a catalyst for performing a metal mediated cross-coupling reaction. In certain embodiments, the kit further comprises a peptide coupling reagent and a catalyst for performing a metal mediated cross-coupling reaction.

In certain embodiments, the peptide coupling reagent is selected from the group consisting of dicyclohexylcarbodiimide, diisopropylcarbodiimide, HATU, HOAt, HBTU, TBTU, HOBt, HCTU, BOP, PyAOP, and T3P; or the peptide coupling reagent is a combination of any the foregoing.

In certain embodiments, the catalyst for performing a metal mediated cross-coupling reaction is a catalyst for performing a Buchwald-Hartwig coupling, a Sonogashira coupling, a Heck coupling, a Negishi coupling, a Stille coupling, a Suzuki coupling, a Hiyama coupling, a Fukuyama coupling, an Ullmann coupling, or a Chan-Lam coupling. In certain embodiments, the catalyst for performing a metal mediated cross-coupling reaction is a catalyst for performing a Buchwald-Hartwig coupling. In certain embodiments, the catalyst for performing a metal mediated cross-coupling reaction comprises Xantphos, Xantphos Pd G2, Xantphos Pd G3, Xantphos Pd G4, tBuXphos, tBuXPhos Pd G1, tBuXPhos Pd G2, tBuXPhos Pd G3, tBuXPhos Pd G4, XPhos Pd G1, XPhos Pd G2, XPhos Pd G3, XPhos Pd G4, tBuBrettPhos, tBuBrettPhos G2, BuBrettPhos G3, RuPhos Pd G1, RuPhos Pd G2, RuPhos Pd G3, RuPhos Pd G4, SPhos, SPhos Pd G1, SPhos Pd G2, SPhos Pd G3, SPhos Pd G4, BrettPhos, BrettPhos Pd, BrettPhos Pd G2, BrettPhos Pd G3, BrettPhos Pd G4, JohnPos, DavePhos, AlPhos, CPhos, CPhos Pd G1, CPhos Pd G2, CPhos Pd G3, QPhos, QPhos Pd G1, QPhos Pd G2, QPhos Pd G3, EPhos, EPhos Pd G1, EPhos Pd G2, EPhos Pd G3, EPhos Pd G4, APhos, APhos Pd G1, APhos Pd G2, APhos Pd G3, APhos Pd G4, JackiePhos, JackiePhos Pd G1, JackiePhos Pd G2, JackiePhos Pd G3, MorDalphos, MorDalphos Pd G1, MorDalphos Pd G2, MorDalphos G3, MePhos, HandaPhos, TrixiePhos, TrixiePhos Pd G1, TrixiePhos Pd G2, TrixiePhos Pd G3, TrixiePhos Pd G4, Josiphos, CyJohnPhos, CyJohnPhos Pd G1, CyJohnPhos Pd G2, CyJohnPhos Pd G3, CyJohnPhos Pd G4, EvanPhos, RockPhos, Rock Phos Pd G2, Rock Phos Pd G3, AdBrett Phos, AdBrett Phos Pd, AdBrett Phos Pd G2, AdBrett Phos Pd G3, VPhos, VPhos Pd G1, VPhos Pd G2, VPhos Pd G3, or VPhos Pd G4.

In another aspect, the present disclosure provides methods of making a conjugate disclosed herein, wherein X is a small molecule or a plurality of small molecules; and D is an encoding peptide tag; comprising:
i) providing a conjugate disclosed herein, wherein X is halo, protected amino, or protected carboxy, and D is hydrogen;
ii) contacting the conjugate with an encoding amino acid disclosed herein or a spacing monomer of disclosed herein and a peptide coupling reagent;
iii) optionally repeating step ii);
iv) protecting the N-terminus or C-terminus of the encoding peptide tag;
v) deprotecting X;
vi) contacting the conjugate with:
   a) a small molecule comprising a carboxy moiety or an amino moiety and a peptide coupling reagent; or
   b) a small molecule comprising a halo moiety and a catalyst for performing a metal mediated cross-coupling reaction;
vii) optionally repeating step vi); and
viii) deprotecting the N-terminus or C-terminus of the encoding peptide tag, thereby providing a conjugate wherein X is a small molecule or a plurality of small molecules, and D is an encoding peptide tag.

In certain embodiments, step ii) further comprises purifying the conjugate.

In certain embodiments, step iv) comprises protecting the N-terminus of the encoding peptide tag. In certain embodiments, step iv) further comprises purifying the conjugate.

In certain embodiments, step v) further comprises purifying the conjugate.

In certain embodiments, step vi) further comprises purifying the conjugate after step a) or b).

In certain embodiments, step viii) comprises deprotecting the N-terminus of the encoding peptide tag. In certain embodiments, step viii) further comprises purifying the conjugate.

In certain embodiments, step iii) is performed. In certain embodiments, step iii) is performed 2, 3, 4, 5, 6, 7, 8, 9, or 10 times.

In certain embodiments, step vii) is performed. In certain embodiments, step vii) is performed 2, 3, 4, 5, 6, 7, 8, 9, or 10 times.

In another aspect, the present disclosure provides methods of making a conjugate disclosed herein, wherein X is a small molecule or a plurality of small molecules; and D is an encoding peptide tag; comprising:
I) providing a conjugate disclosed herein, wherein X is halo, amino, or carboxy, and D is a protected carboxy or protected amino;
II) contacting the conjugate with:
   a) a small molecule comprising a carboxy moiety or an amino moiety and a peptide coupling reagent; or
   b) a small molecule comprising a halo moiety and a catalyst for performing a metal mediated cross-coupling reaction;
III) optionally repeating step II);
IV) deprotecting D;
V) contacting the conjugate with an encoding amino acid of disclosed herein or a spacing monomer of disclosed herein and a peptide coupling reagent, thereby yielding a conjugate, wherein X is a small molecule or a plurality of small molecules; and D is an encoding peptide tag; and
VI) optionally repeating step V).

In certain embodiments, step II) further comprises purifying the conjugate after step a) or b).

In certain embodiments, step IV) further comprises purifying the conjugate.

In certain embodiments, step V) further comprises purifying the conjugate.

In certain embodiments, step VI) further comprises purifying the conjugate.

In certain embodiments, step III) is performed. In certain embodiments, step III) is performed 2, 3, 4, 5, 6, 7, 8, 9, or 10 times.

In certain embodiments, step VI) is performed. In certain embodiments, step VI) is performed 2, 3, 4, 5, 6, 7, 8, 9, or 10 times.

In certain embodiments, the peptide coupling reagent is selected from the group consisting of dicyclohexylcarbodiimide, diisopropylcarbodiimide, HATU, HOAt, HBTU, TBTU, HOBt, HCTU, BOP, PyAOP, and T3P; or the peptide coupling reagent is a combination of any the foregoing.

In certain embodiments, the catalyst for performing a metal mediated cross-coupling reaction is a catalyst for performing a Buchwald-Hartwig coupling, a Sonogashira coupling, a Heck coupling, a Negishi coupling, a Stille coupling, a Suzuki coupling, a Hiyama coupling, a Fukuyama coupling, an Ullmann coupling, or a Chan-Lam coupling. In certain embodiments, the catalyst for performing a metal mediated cross-coupling reaction is a catalyst for performing a Buchwald-Hartwig coupling. In certain embodiments, the catalyst for performing a metal mediated cross-coupling reaction comprises Xantphos, Xantphos Pd G2, Xantphos Pd G3, Xantphos Pd G4, tBuXphos, tBuXPhos Pd G1, tBuXPhos Pd G2, tBuXPhos Pd G3, tBuXPhos Pd G4, XPhos Pd G1, XPhos Pd G2, XPhos Pd G3, XPhos Pd G4, tBuBrettPhos, tBuBrettPhos G2, BuBrettPhos G3, RuPhos Pd G1, RuPhos Pd G2, RuPhos Pd G3, RuPhos Pd G4, SPhos, SPhos Pd G1, SPhos Pd G2, SPhos Pd G3, SPhos Pd G4, BrettPhos, BrettPhos Pd, BrettPhos Pd G2, BrettPhos Pd G3, BrettPhos Pd G4, JohnPos, DavePhos, AlPhos, CPhos, CPhos Pd G1, CPhos Pd G2, CPhos Pd G3, QPhos, QPhos Pd G1, QPhos Pd G2, QPhos Pd G3, EPhos, EPhos Pd G1, EPhos Pd G2, EPhos Pd G3, EPhos Pd G4, APhos, APhos Pd G1, APhos Pd G2, APhos Pd G3, APhos Pd G4, JackiePhos, JackiePhos Pd G1, JackiePhos Pd G2, JackiePhos Pd G3, MorDalphos, MorDalphos Pd G1, MorDalphos Pd G2, MorDalphos G3, MePhos, HandaPhos, TrixiePhos, TrixiePhos Pd G1, TrixiePhos Pd G2, TrixiePhos Pd G3, TrixiePhos Pd G4, Josiphos, CyJohnPhos, CyJohnPhos Pd G1, CyJohnPhos Pd G2, CyJohnPhos Pd G3, CyJohnPhos Pd G4, EvanPhos, RockPhos, Rock Phos Pd G2, Rock Phos Pd G3, AdBrett Phos, AdBrett Phos Pd, AdBrett Phos Pd G2, AdBrett Phos Pd G3, VPhos, VPhos Pd G1, VPhos Pd G2, VPhos Pd G3, or VPhos Pd G4.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g., “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O— alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

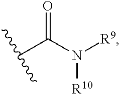

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

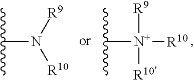

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

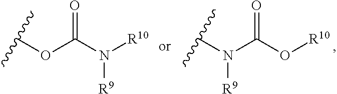

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two, three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated, and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene, and adamantane. Exemplary fused carbocycles include decalin, naphthalene, 1,2,3,4-tetrahydronaphthalene, bicyclo[4.2.0]octane, 4,5,6,7-tetrahydro-1H-indene, and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl, and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “heteroalkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

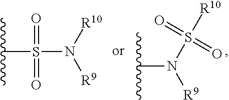

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

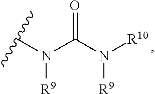

wherein R⁹ and R¹⁰ independently represent hydrogen or a hydrocarbyl.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The term “protecting group” refers to a reversibly formed derivative of an existing functional group in a molecule. The protecting group is temporarily attached to decrease reactivity so that the protected functional group does not react under synthetic conditions to which the molecule is subjected in one or more subsequent steps. Exemplary protecting groups are disclosed in Greene's Protective Groups in Organic Synthesis, Wiley, 5^{th} edition, Oct. 27, 2014, the contents of which is incorporated fully by reference herein.

### Examples

The invention now being generally described, it will be more readily understood by reference to the following examples, which are included merely for purposes of illustration of certain aspects and embodiments of the present invention, and are not intended to limit the invention.

### Example 1: Preparation of Exemplary PELs

### Material & Methods

Monosized Polystyrene M NH₂ resin (20 μm, HM12002) was purchased from Rapp Polymere. Fmoc-amino acids and Rink Amide were purchased from Novabiochem, Millipore Sigma, Chem-Impex Int′l Inc, Advanced ChemTech, or BroadPharm. Trt-Val-OH was prepared according to a literature procedure. Fmoc-Seramox(Boc, tBu)-OH (Fmoc-Smx(Boc, tBu)-OH) was prepared from commercial 2-(Fmoc-amino)acetaldehyde and H-Ser(tBu)-OBzl·HCl through one-pot reductive amination and Boc protection, followed by Pd/C catalyzed hydrogenation. Trifunctional building blocks used in library synthesis were purchased from Enamine. Anilines, boronic acids, and carboxylic acid building blocks were obtained from various commercial suppliers and used without further purification. 1-[Bis(dimethylamino)methylene]-1H-1,2,3-triazolo[4,5-b]pyridinium 3-oxide hexafluorophosphate (HATU, >97%) was purchased from P3 Biosystems. OmniSolv® grade N,N-dimethylformamide (DMF) was purchased from Millipore Sigma and treated with 1 AldraAmine trapping stick per 4 L before use. Biotech-grade N,N-diisopropylethylamine (DIPEA) and anhydrous tetrahydrofuran (THF) were dispensed from a solvent distillation system before use. HPLC-grade dichloromethane (DCM) and acetonitrile (MeCN), and ACS grade diethyl ether were purchased from Millipore Sigma.

The remaining chemicals were purchased in purities of ≥95% and used without further purification. Piperidine, trifluoroacetic acid (TFA), ethanedithiol (EDT), triisopropylsilane (TIPS), phenylsilane, triphenylmethyl chloride (Trt-Cl), and Pd(PPh₃)₄ were purchased from Millipore Sigma. Guanidine hydrochloride was ordered from Thermo Fisher Scientific. Sodium diethyldithiocarbamate was bought from Alfa Aesar. (+)-JQ-1 (free acid) was ordered from Cayman Chemical. N10-(trifluoroacetyl)pteroic acid was purchased from Biosynth Carbosynth. XPhos was obtained as a gift from Sigma-Aldrich. AlPhos was prepared according to literature procedures. G4 Pd XPhos was purchased from Sigma-Aldrich or prepared according to literature procedure. Water was deionized by a MilliQ water purification system from Millipore. 10× Phosphate buffered saline Corning® was ordered from VWR, diluted with MilliQ water to 1×, and the pH was adjusted to 7.4.

Biotinylated Human FOLR1 Protein, Fc,Avitag™ (FO1-H82F9) was purchased from Acro Biosystems. BRD4(1)[42-168]-Gln(biotin) was prepared following procedures reported by Hartrampf et al.

Supelclean™ LC-18 SPE Tubes, empty polypropylene SPE tubes with 20 μm polyethylene frits for SPPS, and C18 ZipTips (0.6 μL) were purchased from Millipore Sigma. A flash purification system (Biotage Selekt) was used to purify model conjugates with a Biotage Sfär C18 column (12 g, 20 μm particle size, 300 Å pore size).

Mass analysis was performed on an Agilent 6545 Q-TOF LC-MS system. Mobile phases A (0.1% formic acid (FA) in water) and B (0.1% FA in MeCN) were prepared from LC-MS grade LiChrosolv® water and MeCN from Millipore Sigma, and Optima™ LC/MS grade formic acid from Thermo Fisher Scientific. Samples (approx. 10 ng in 5% MeCN in water+0.1% TFA) were analyzed on a Zorbax 300SB—C3 column (2.1×150 mm, 5 μm, flow rate=0.8 mL/min) using the following gradient: 0-2 min 1% B, 2-11 min 1-91% B, 11-12 min 91-95% B. 12-15 min 95% B. MS were recorded from 4-12 min.

nLC-MS/MS was performed on an EASY-nLC 1200 nano liquid chromatography system connected to an Orbitrap Fusion Eclipse Tribrid Mass Spectrometer equipped with a Nanospray Flex ion source and an Acclaim™ PepMap™ RSCL column (50 μm×15 cm, nanoViper, PN 164943) and an Acclaim™ PepMap™ 100 trap column (75 μm×2 cm, nanoViper, PN 164946), which were all purchased from Thermo Fisher Scientific. Mobile phase A (0.1% FA in water) and B (0.1% FA in 80% MeCN and 19.9% water) were prepared with LiChrosolv® water and MeCN suitable for MS from Millipore Sigma, and Optima™ LC/MS grade formic acid from Thermo Fisher Scientific. Positive ion spray voltage was set to 2000-2300 V during instrument tune.

Enrichment of model tags was performed in an automated Kingfisher Duo Prime Purification system for handling of magnetic beads (Thermo Fisher Scientific) equipped with a 12-rod magnetic arm for application in 96-well plates. 1 mg of Dynabeads™ MyOne™ Streptavidin T1 (invitrogen) were used per condition in phosphate-buffered saline (PBS, pH 7.4), wash buffer (PBS+10% fetal bovine serum (FBS)+0.02% Tween 20), or 6 M Guanidine HCl in 200 mM phosphate buffer (pH 6.8).

De Novo Sequencing by nLC-MS/MS

Samples (5 μL) were analyzed using a standard gradient (1-31% B in A over 50 min, then 31-91% B in A for 10 min, 300 nL/min, with A=water+0.1% FA and B=80% MeCN in water+0.1% FA). Primary MS (application mode=peptide) was detected in the orbitrap using the following parameters: resolution=120000, mass range=normal, quadrupole isolation, scan range=280-1800 m/z, RF lens=30%, AGC target=250%, maximum injection time=auto, 1 microscan, data type=profile, polarity=positive. The following filters were applied for precursor selection: monoisotopic precursor selection=peptides, precursor selection range=280-1800 m/z, intensity threshold=4.0e4, charge states=2-10, dynamic exclusion (exclusion after 2n within 30 s, mass tolerance=10 ppm), targeted mass (mass list for targeted inclusion were generated by calculation of unique masses of encoding tags for every library), time between master scans=3 s. Fragmentation was induced by higher-energy collisional dissociation (HCD) and electron-transfer dissociation with higher-energy collision (EThcD). Specifications HCD: isolation mode=quadrupole, isolation window=1.3 m/z, isolation offset=off, activation type=HCD, collision energy mode=assisted (15, 30, 45%), detection=orbitrap, resolution=50000, mass range=normal, first mass=120 m/z, normalized AGC target=300%, maximum injection time=86 s, 2 microscans, data type=centroid. Specifications EThcD: isolation mode=quadrupole, isolation window=1.3 m/z, isolation offset=2 Da, activation type=ETD, use calibrated charge-dependent ETD parameters, ETD supplemental activation=EThcD, SA collision energy=25%, detection=orbitrap, resolution=50000, mass range=normal, first mass=120 m/z, normalized AGC target=300%, maximum injection time=86 s, 2 microscans, data type=centroid.

### Exemplary Synthetic Procedures

HATU coupling: Amino acid (5 equiv.) was dissolved in HATU (0.38 M in DMF, 4.5 equiv.). DIPEA (15 equiv.) was added, and the resulting solution was immediately added to a fritted syringe charged with the amine coupling partner on resin. After 15 min, the reaction solution was removed by vacuum filtration, and the resin was washed with DMF (3×).

Fmoc deprotection: To a fritted syringe charged with Fmoc-protected amine on resin 20% piperidine in DMF was added, and after 5 min the reaction solution was removed by vacuum filtration (2×). The resin was washed with DMF (3×).

Trt deprotection: Trt-protected α-amine on resin in a fritted syringe was washed with DCM (3×). DCM/TIPS/TFA (97/2/1) was added, and after 2 min the reaction solution was removed by vacuum filtration (5×). The resin was washed with DCM (3×) and DMF (3×). Alloc deprotection: Alloc-protected amine on resin in a fritted syringe was washed with DCM (3×). A freshly prepared DCM solution of Pd(PPh₃)₄ (0.1 equiv.) and phenylsilane (10 equiv.) was added, and after 20 minutes the reaction solution was removed by vacuum filtration (2×). The resin was washed with DCM (3×), DMF (3×), sodium diethyldithiocarbamate (0.5% in DMF, 2×5 min), and DMF (3×).

Trt protection: Free amine on resin in a fritted syringe was washed with DCM (3×). A DCM solution with Trt-Cl (5 equiv.) and DIPEA (15 equiv.) was added, and after 1 h the reaction solution was removed by vacuum filtration (2×). The resin was washed with DCM (3×) and DMF (3×).

Alloc protection: Free amine on resin in a fritted syringe was washed with DCM (3×). A DCM solution with allyl chloroformate (5 equiv.) and DIPEA (15 equiv.) was added, and after 1 h the reaction solution was removed by vacuum filtration (2×). The resin was washed with DCM (3×) and DMF (3×).

### Synthesis of Model Conjugates

H-Lys-Cha-Mox-Val-Dmf-Pro-Lys-Thr-Tyr-Leu-Val-Smx-Lys(Alloc)-Rink amide was prepared on 20 μm PS resin on a 20 μmol scale by standard Fmoc/tBu solid-phase peptide synthesis as outlined in “Exemplary Synthetic Procedures”. Seramox (2 equiv.) was coupled using HATU (1.9 equiv.) and DIPEA (6 equiv.) in DMF for 60 min at rt. After assembly of the encoding peptide, a Trt protecting group was introduced to the free N-terminus, followed by removal of the Alloc protecting group from the C-terminal Lys. Fmoc-PEG2-OH (5 equiv.) was coupled with HATU (4.5 equiv.) and DIPEA (15 equiv.) in DMF for 15 min at rt as a linker between the small molecules and the encoding tag. (+)-JQ-1 (2 equiv.) was coupled to the free amine using HATU (1.9 equiv.) and DIPEA (6 equiv.) in DMF for 60 min at rt. Folic acid was introduced to the encoding tag following reported procedures. Assembled model conjugates were cleaved from the resin using TFA/H₂O/EDT/TIPS (94:2.5:2.5:1) for 2 h at rt and isolated by repeated precipitation and centrifugation with cold diethyl ether (3×). Purification by reverse-phase column chromatography (C18 silica) afforded pure model conjugates.

### Enrichment of Model Conjugates

The following protocol steps were performed using an automated KingFisher Magnetic Beads handling system at room temperature unless indicated otherwise, with each step corresponding to a fresh well of a 96-well plate:
1. Bead rinse: 1 mL wash buffer, release beads, 30 s mixing at medium speed, collect 3×
2. Bead rinse: 1 mL wash buffer, release beads, 30 s mixing at medium speed, collect 3×
3. Conjugation of protein to streptavidin: biotinylated protein (312 μM) in 0.5 mL wash buffer, 20 sec release beads at medium speed, 30 min mixing at slow speed, collect 5× (1 s)
4. Protein wash: 1 mL wash buffer, release beads, 30 s mixing at medium speed, collect 3×
5. Protein wash: 1 mL wash buffer, release beads, 30 s mixing at medium speed, collect 3×
6. Model conjugate binding (at 10° C.): model conjugate (200, 500, or 1000 μM) in 0.2 mL PBS+10% FBS, 20 sec release beads at medium speed, 60 min mixing at slow speed, collect 5× (1 s)
7. Peptide wash: 0.5 mL PBS, release beads, 10 s mixing at medium speed, collect 2×
8. Peptide wash: 0.5 mL PBS, release beads, 10 s mixing at medium speed, collect 2×
9. Elute: 100 μL 6 M Guanidine HCl, release beads, 1 min mixing at fast speed, collect 5× (1 s)
10. Elute: 100 μL 6 M Guanidine HCl, release beads, 1 min mixing at fast speed, collect 5× (1 s)

Solutions from wells 9 and 10 were combined for oxidative cleavage. 6.5 μL of 1 mM NaIO₄ in PBS was added, and Smx cleavage was performed over 30 min in the dark at rt. Alternatively, steps 9 & 10 in the KingFisher protocol plus the oxidative cleavage described in the previous sentence can be replaced through a single KingFisher protocol step wherein oxidative cleavage is achieved as step 9 as follows: 40° C., 0.1 mL 65 μM NaIO4 in PBS, release beads, 30 min mixing at medium speed, collect 5× (1 s). The reaction was quenched by 6.5 μL of 0.1 M Na₂SO₃ for 30 min in the dark at rt. Samples were desalted using C18 ZipTips, eluted in 100 mM Guanidine HCl in 70% MeCN in H₂O+0.1% FA, and lyophilized or dried by spin vacuum. Dried samples were resuspended in 6 μL water+0.1% FA, and 5 μL were injected for analysis by nLC-MS/MS. Alternatively, the samples can be directly eluted into eluted in 4 μL of MeOH/H2O/FA (49.5/49.5/1) FA, and diluted with 6 μL H2O+0.1% FA. 5 μL of the prepared solutions were injected for analysis by nLC-MS/MS.

### Exemplary Small Molecule Library Synthesis Procedures

Carboxylic Acid Coupling: Carboxylic acid (10 equiv.) was dissolved in HATU (0.38 M in DMF, 4.5 equiv.). DIPEA (15 equiv.) was added, and the resulting solution immediately added to a fritted syringe charged with the amine coupling partner on DMF-soaked resin. After 1 h, the reaction solution was removed by vacuum filtration, and the resin was washed with DMF (3×). The procedure was repeated once.

Pd-mediated C—C cross-coupling: A 1.5 mL microcentrifuge tube was charged with aryl bromide on dry resin and the corresponding boronic acid (5 equiv.). A freshly prepared solution of Pd G4 XPhos (1.2 equiv.) and XPhos (1.2 equiv.) in degassed THE and K₃PO₄ (0.5 M in degassed water, 5 equiv.) were added (final concentration 10 mM). The microcentrifuge tubes were quickly purged with N₂, sealed, and agitated on a nutating mixer for 24 h. The reaction mixture was transferred into a fritted syringe and washed with DCM (3×), DMF (2×), water (2×), DMF (3×), sodium diethyldithiocarbamate (0.5% in DMF, 3×10 min), and DMF (3×).

Pd-mediated C—N cross-coupling: A 2 mL HPLC vial was charged with aryl bromide on dry resin, [(AlPhos)₂Pd₂(cod)](0.6 equiv.), and aniline (if solid, 3 equiv.). The vial was brought into a glovebox under N₂ atmosphere. 2-MeTHF (final concentration 10 mM), aniline (if liquid, 3 equiv.), and DBU were added. The vial was capped, removed from the glovebox and agitated in an incubator at 200 rpm and 50° C. for 2 h. The reaction mixture was transferred into a fritted syringe and washed with DCM (3×), DMF (2×), water (2×), DMF (3×), sodium diethyldithiocarbamate (0.5% in DMF, 3×1 h min), and DMF (3×).

### Exemplary Library Synthesis

Monosized PS resin (305 mg, 400 μmol) in a fritted 20 mL syringe was sequentially coupled to Rink amide, and Fmoc-Lys(Alloc)-OH using the standard HATU coupling and Fmoc deprotection procedures. Fmoc-Smx(Boc, tBu)-OH (2.5 equiv.) was coupled using HATU (0.38 M in DMF, 2.3 equiv.) and DIPEA (8 equiv.) for 1 h. Trt-Val-OH was coupled using the standard HATU coupling procedure with 1 h reaction time. The resin was subjected to the standard Alloc deprotection procedure and evenly split into 12 fritted syringes. An amino acid building block was coupled to each fraction using the standard HATU coupling procedure. Each fraction was subjected to the standard Fmoc deprotection, Alloc protection, and trityl deprotection procedure. The corresponding encoding monomer was coupled using the standard HATU coupling procedure. LC-MS analysis of each fraction showed a single major peak corresponding to the expected product. The resin was pooled in a fritted 20 mL syringe, subjected to the standard Fmoc deprotection procedure, and split into 18 fritted 3 mL syringes. Each fraction was sequentially coupled to the two corresponding encoding monomers followed by Fmoc-Lys(Boc)-OH using the standard HATU coupling and Fmoc deprotection procedures. The free N-terminus was trityl protected. Each fraction was Alloc deprotected using the standard procedure and functionalized with the corresponding trifunctional building block (3 equiv.) using HATU (0.38 M in DMF, 2.7 equiv.) and DIPEA (9 equiv.) for 2 h. The resin was pooled, subjected to standard Fmoc deprotection and split into 62 fractions. Each fraction was subjected twice to coupling of the corresponding carboxylic acid building block (10 equiv.) using HATU (0.38 M in DMF, 9 equiv.) and DIPEA (30 equiv.) for 1 h. Each fraction was washed with DCM (3×), treated with acetyl chloride (5 equiv.) and DIPEA (15 equiv.), and washed with DCM (3×) and DMF (3×). Standard trityl deprotection was followed by introduction of the corresponding encoding monomers using the standard HATU coupling and Fmoc deprotection procedures. The fractions were pooled, Fmoc deprotected and subjected to standard HATU coupling using Trt-Val-OH and a 1 h reaction time. The total amount of resin was split into two parts to enable the synthesis of two different libraries. Each part was split into 35 parts. Each fraction was subjected to the standard C—C and C—N cross-coupling procedures using the corresponding boronic acids and anilines, respectively. Each fraction was Fmoc deprotected using the standard procedure and coupled to the corresponding encoding monomers using the standard HATU coupling and Fmoc deprotection procedures. Each library was pooled separately, and coupled to either Fmoc-Lys(Boc)-OH or Fmoc-Arg(Pbf)-OH using the standard HATU coupling and Fmoc deprotection procedures. The libraries were cleaved from resin using TFA/H₂O/EDT/TIPS (94:2.5:2.5:1) for 2 h at rt and isolated by repeated precipitation and centrifugation with cold diethyl ether (3×). Purification by solid phase extraction afforded the final peptide-encoded small molecule libraries.

### Results and Discussion

The first fundamental step in the implementation of PELs was the identification of a suitable resin. Different monosized resins were surveyed with varying chemical composition and ultimately it was found that polystyrene (PS) resin (20 μm) was most suited for the application. Tentagel resin used in previous peptide libraries also fulfilled requirements for high stability under various reaction conditions, but high degrees of PEG leakage during resin cleavage hampered analysis and purification. The relatively small PS exhibited lower swelling properties, which—while still compatible with the desired reactions—necessitated specialized filters with smaller pore sizes during solid phase synthesis.

The resin is functionalized with the commonly available Fmoc-Rink amide (RA) linker, which undergoes cleavage from the resin under established strongly acidic conditions (TFA/H₂O/EDT/TIPS, 94:2.5:2.5:1). These cleavage conditions were validated to be compatible with chemically diverse small molecules. Fmoc-Lys(Alloc)-OH was coupled to RA to serve as a branching linker with orthogonally protected amines for separate synthetic functionalization (FIG. 2). The Fmoc-protected α-amino group is selectively deprotected and coupled with Seramox (Fmoc-Smx(Boc, tBu)-OH, a serine derivative which can be cleaved under oxidizing conditions. This linker serves as anchor for the encoding peptide, which requires cleavage from the conjugate prior to nLC-MS/MS analysis. A salient feature of Smx is that oxidative cleavage furnishes a basic amine on the C-terminus of the peptide tag, which results in increased sensitivity during MS detection. Importantly, the two cleavable linkers are fully orthogonal.

The Fmoc-protected amine of Smx is selectively deprotected and coupled with an amino acid (AA) featuring a trityl protecting group. This allows for introduction of a protecting group stable during subsequent Alloc deprotection of the F-amine of the branching Lys linker, but also functions as the first encoding monomer. Starting with the same encoding monomer for a whole library allows for higher stringency during later data processing and higher confidence during sequencing. The fundamental framework has thus been completed, offering two fully orthogonal protecting groups for further synthetic elaboration during parallel combinatorial synthesis as well as two orthogonally cleavable linkers.

The peptidic encoding site can be either Fmoc or Trt protected, allowing for standard deprotection protocols and peptide coupling protocols to propagate the encoding chain. Either protecting group can be chosen in order to accommodate the respective small molecule chemistry of a subsequent step.

### Identification of Suitable Encoding Monomers

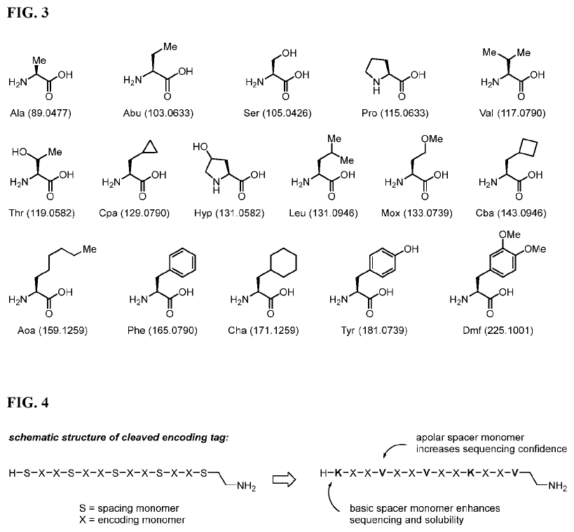

Any encoding strategy relies on a type of alphabet in which information is spelled out, i.e., encoded. The letters of such an alphabet are represented by encoding monomers, which in the case of PELs are amino acids. A single building block of the small molecule can be represented by one or more encoding monomers, thus exponentially increasing the theoretical amount of possible encoded building blocks per combinatorial synthetic step. 16 monomers with unique exact masses were chosen on the basis of anticipated chemical inertness under a variety of reaction conditions (FIG. 3). Accordingly, the selected monomer set features primarily aliphatic and ether substituents. Moderate hydrophilicity to accommodate for later solubility and prevention of unspecific binding is achieved through protected alcohol substituents. The use of monomers with a relatively high molecular weight ultimately affords encoding tags with a higher average weight, which allows to vary isolation width of quadrupole filtering during mass spectrometric analysis leading to an improved signal-to-noise ratio. None of the encoding monomers are expected to be charged during mass spectrometric analysis, giving a narrow and well controlled distribution of charged states of the encoding peptide. However, controlled inclusion of basic monomers to achieve a pre-defined charge state is expected to further increase hydrophilicity and ion fragmentation, the latter of which in turn enables a higher confidence during de novo sequencing.

### Encoding Tag Design

The encoding tag consists of a polypeptide wherein one or a combination of amino acids encode a single small molecule building block. With 16 encoding monomers selected, a combination of two amino acids would allow for the incorporation of up to 256 building blocks during combinatorial synthesis. Increasing the number of encoding monomers or the length of the encoding subunit could readily allow for larger sets of building blocks to be used during small molecule diversification.

As a synthetic requirement of the encoding tag, the polypeptide built from non-reactive monomers can be selectively elongated under parallel combinatorial synthesis. Additionally, the features of the encoding tag have a central influence on the ability to perform de novo sequencing by MS/MS, and several crucial parameters were identified. In order to achieve higher confidence in sequenced tags, every tag ends with the same monomer (spacer monomer). As described in the synthetic framework, this monomer is bound to Smx, thus, affording an ethylamine on the C-terminus of the peptide tag after oxidative cleavage. Such repeating spacer monomers are incorporated throughout the polypeptide tag in order to further increase confidence in sequenced tags but also to facilitate reproducible protecting group incorporation. The encoding peptide is either terminated with an Fmoc or Trt protecting group, depending on the subsequent small molecule functionalization reaction conditions. By terminating each encoding subunit with the same amino acid, this new amino acid can directly be introduced with a suitable protecting group.

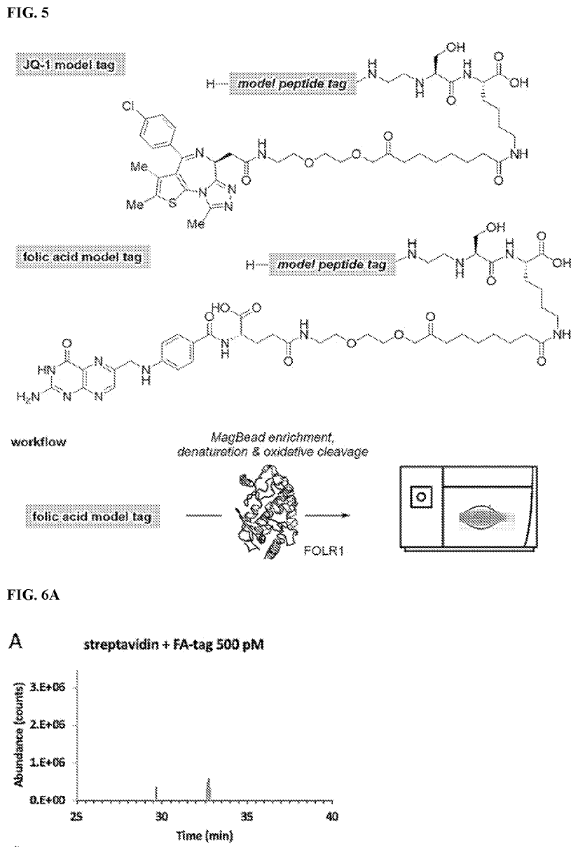

Proteomic data analysis and insight into peptide fragmentation suggests that at least one basic residue on the C-terminus increases de novo sequencing quality. Similar enhancement is observed by inclusion of basic monomers as repeating spacers throughout the tag (FIG. 4). Additionally, polar amino acids can be included to adjust the polarity of the tag structure, influencing solubility and chromatographic separation (FIG. 4B). Since these monomers are the same at the same position for every library member, they also serve as an additional sequencing control to further increase confidence in hits. Moreover, the inclusion of several basic monomers alleviates some of the hydrophobicity impaired by the conjugated small molecules and the otherwise mostly aliphatic monomers. Importantly, the total amount of basic residues is well defined, affording a predictable and uniform charge distribution during MS/MS analysis. Incorporation of basic residues (Lys, Arg, homo-Arg, ornithine, diaminobutyric acid, etc.) or apolar monomers (Ala, Val, etc.) at the N-terminus can—in addition to enhancing solubility—serve as the identifying tag of a library, thus allowing multiplexed analysis of mixed libraries.

High-confidence sequencing information can be extracted from peptides with chain lengths of 9-15 amino acids. Our encoding strategy uses subunits of up to two amino acids to encode each small molecule building block with defined spacer monomers preceding every encoding subunit. Thus, the encoding tag of a library with 4 building blocks would consist of 13 amino acids. The length of the encoding tag can be reduced if a smaller subset of a building block requires only one encoding monomer or one spacing monomer can be avoided, still leaving the tag in a suitable range for de novo sequencing.

### MS/MS-sequencing

High-fidelity sequencing is required to reliably decode encoding tags and identify hits of affinity selection. A robust chromatographic separation of the encoding tags permits to use MS duty cycles of 3 seconds. Within these time intervals, a primary MS is recorded in the orbitrap. A range of filters (monoisotopic precursor selection, charge state, m/z range, dynamic exclusion) is applied to select precursors for fragmentation and reduce background. Additionally, a targeted mass inclusion filter can be applied to select peptides corresponding to the library tag design. Highest average confidence and sequence recovery was observed by prioritizing electron-transfer dissociation with supplemental activation by higher-energy collision dissociation (EThcD). The former leads to fragmentation of the precursor into c and z ions, whereas the latter leads to b and y ions, providing additional sequence information. Another spectrum with fragmentation by higher-energy collision dissociation (HCD) with normalized collision energy of 25% is acquired of the same precursor with a lower priority if additional time is available in the duty cycle.

Sequences are decoded by the automated de novo sequencing software package PEAKS. Non-canonical amino acids and C-terminal ethylamine are defined as post-translational modifications (PTMs) and used to extract amino acid sequence information from raw mass spectra. Sequences are further refined by data filtration using a python script to isolate sequences matching the library design. Further data analysis using z-scores or enrichment fingerprints enables the identification of binding molecules.

### Enrichment & Sequencing of Model Conjugates

To simulate enrichment and recovery of encoded small molecules, model conjugates of encoding tags and small molecules with reported high affinity for their target protein were prepared. Folic acid and JQ-1 were coupled to a model tag (FIG. 5A) to study their enrichment in the presence of their target proteins, the folate receptor 1 (FOLR1) and bromodomain-containing protein 4 bromodomain-1 (BRD4(1)), respectively. The biotinylated proteins were immobilized on magnetic beads bearing streptavidin for handling during enrichment experiments. The encoded small molecules were incubated with the immobilized target proteins or unloaded streptavidin beads at different concentrations for enrichment. After release of the binding conjugates by protein denaturation and oxidative cleavage, samples were subjected to analysis by nLC-MS/MS (FIG. 5B).

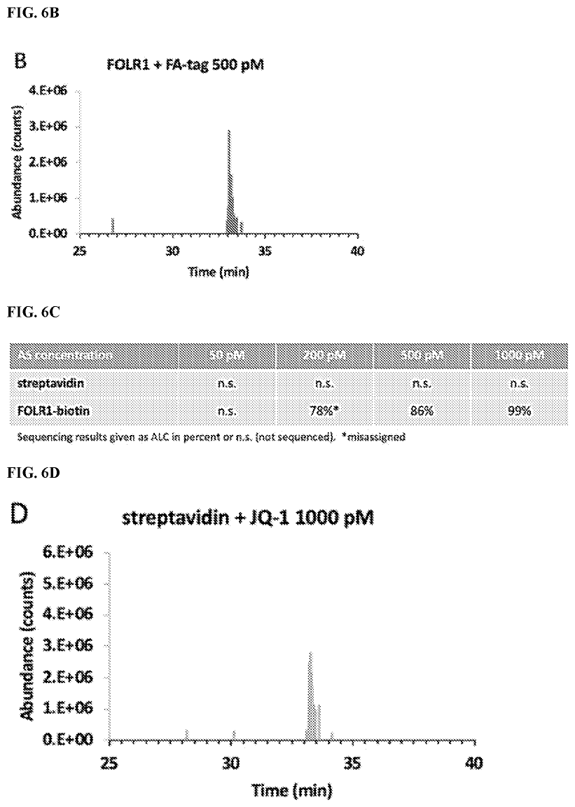

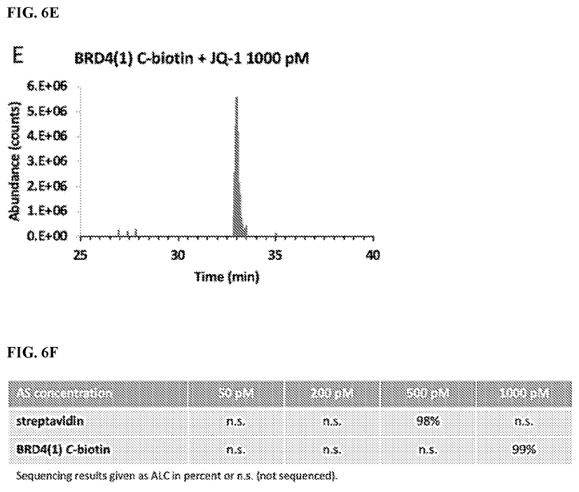

Enrichment of the model conjugate was determined by extracting the corresponding ions from the chromatograms (extracted ion chromatogram (EIC)). The folic acid model conjugate was enriched 6-fold after incubation with FOLR1 as opposed to incubation with unfunctionalized streptavidin beads, FIGS. 6A and 6B, enrichment at 500 μM of the model conjugate). At incubation concentrations of 500 and 1000 μM the encoding tag was correctly sequenced in downstream nLC-MS/MS analysis for samples incubated with FOLR1, indicating a successful workflow at these concentrations (FIG. 6C). Similarly, a twofold enrichment of the model conjugate of JQ-1 was observed after incubation with BRD4(1) in comparison to streptavidin (FIGS. 6D and 6E, enrichment at 1000 μM of the model conjugate). The tag was correctly sequenced at 1000 μM for BRD4(1) with high confidence.

### Implementation of Synthetic Methods

Chemical diversity of the small molecule library is achieved through implementation and development of synthetic methods which are compatible with the on-resin reaction conditions and the encoding tag. In comparison to DNA, the peptide-encoded libraries to display tolerance towards a wide range of reaction conditions. Given the importance of cross-coupling reactions in the synthesis of small molecule therapeutics, a palladium-mediated C—C and C—N bond forming reaction on a model substrate representing a library substrate was implemented.

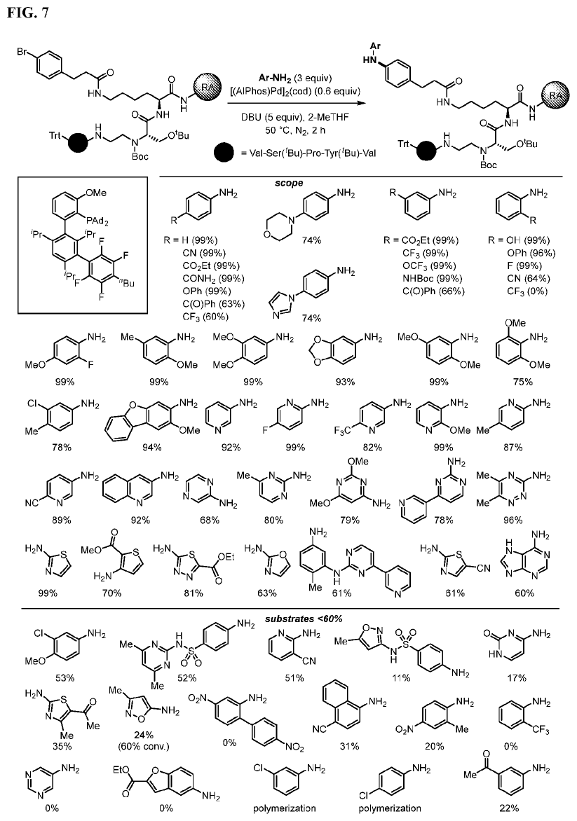

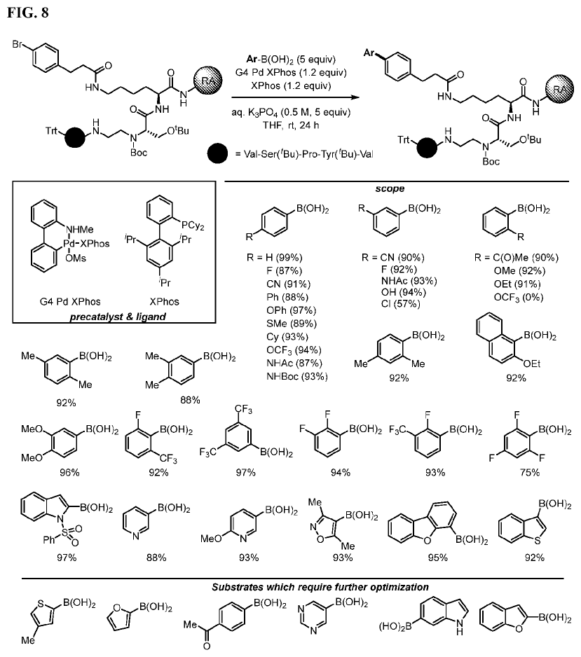

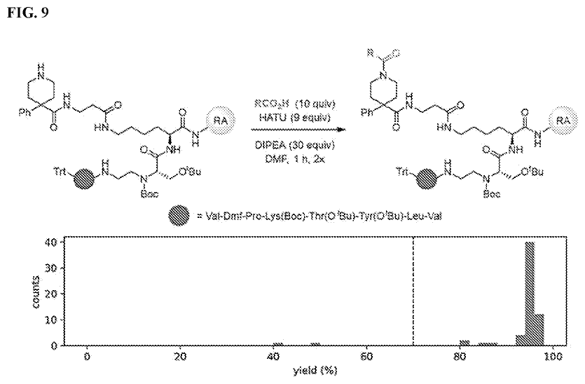

The C—N cross-coupling using biarylphosphine ligated precatalyst in the presence of DBU was found to proceed in high efficiency for a variety of anilines (FIG. 7). A wide range of conditions, including precatalysts, ligands, bases, solvents, temperature, and time, were evaluated. Initially the focuse was on a catalyst system using soluble bases in order to accommodate the resin-bound substrate. As reported previously by Buchwald and co-workers, AlPhos facilitates the use of weak soluble organic bases such as DBU. Use of stochiometric palladium enabled the reaction to proceed efficiently for a wide variety of anilines. Out of 61 randomly selected anilines, 45 coupled with efficiency >60%. The successful substrates feature a large diversity of functional groups including therapeutically privileged heteroaromatic substituents. The C—N bond can be extended to a wider range of amines through the use of different biarylphosphines ligands.

The Suzuki-Miyaura cross-coupling using biarylphosphine-ligated palladium in the presence of aqueous base was found to proceed in high efficiency for a variety of boronic acids. An in-depth evaluation of reaction parameters led to the identification of G4 Pd XPhos together with excess XPhos as the optimal palladium ligand combination. Importantly, the resin was found to have a strong influence on suitable solvents with THE being ideal for PS while MTBE gave better coupling efficiency on Tentagel resin. Out of the wide variety of boronic acids (43) subjected to reaction with the model aryl bromide, only 6 failed to afford the desired product (FIG. 8). These failed substrates are known to suffer from facile in situ protodeborylation.

Carboxylic acids constitute a readily accessible set of building blocks with large chemical diversity which can be introduced through highly efficient amide coupling reactions. To enable high-yielding reactions with between amines and carboxylic acids of varying steric hindrance and reactivity, solid phase synthesis allows for the use of large excess of reagents. Accordingly, 62 carboxylic acids were coupled to a secondary amine model substrate using nine equivalents of HATU twice. 56 carboxylic acids spanning diverse functional groups coupled quantitatively, and only two failed to provide satisfactory conversion or purity.

With several reactions to incorporate a diverse range of coupling partners in hand, central building blocks which would enable sequential functionalization were evaluated. Accordingly, 18 trifunctional building blocks featuring a carboxylic acid, Fmoc-protected amine, and aryl bromide were selected. These three synthetic handles render these substrates well suited as branching building blocks for a combinatorial library, with the carboxylic acid allowing initial coupling to the peptide conjugate. The protected amine can subsequently be functionalized with a wide variety of carboxylic acid building blocks, followed by palladium-mediated cross-coupling reactions of the aryl bromide.

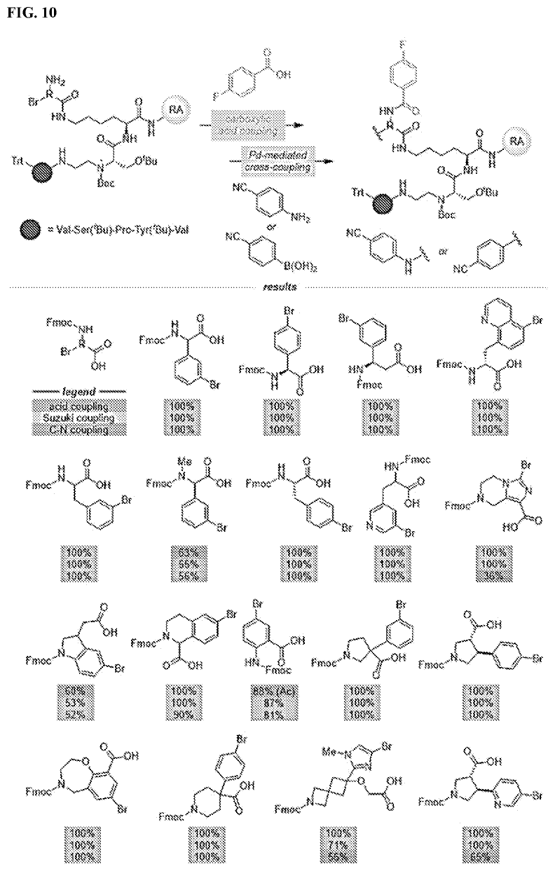

The 18 trifunctional building blocks were coupled to a model peptide conjugate and subjected to amide coupling with a benzoic acid followed by palladium-mediated cross-coupling of a model aniline or boronic acid (FIG. 10). Only one out of 18 trifunctional building blocks did not undergo carboxylic acid coupling, but could be acetylated to prevent undesired reactivity during later synthetic manipulations. Another aniline and a methylated amino acid showed a diminished reactivity towards HATU-mediated carboxylic acid coupling. All substrates efficiently underwent palladium-mediated C—C cross-coupling, whereas for the C—N cross-coupling 3 aryl bromides exhibited attenuated reactivity.

### Synthesis of a Peptide-Encoded Small Molecule Library

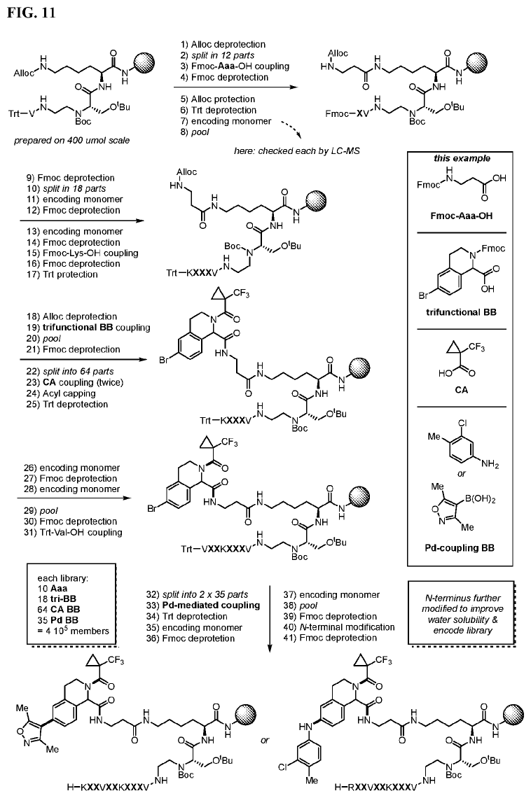

Two peptide-encoded small molecule libraries were prepared using Pd-mediated C—C or C—N bond formation. The synthesis commenced with the large-scale preparation of the orthogonally protected building block featuring an Alloc- and a trityl-amine (FIG. 11). Pd-catalyzed Alloc deprotection was followed by the first split into 12 different fritted syringes. Each fraction was subjected to amide coupling with an Fmoc-protected amino acid. Subsequent two-step protecting group exchange from Fmoc to Alloc enabled continued orthogonal parallel combinatorial synthesis. The trityl protected amine was selectively liberated using weakly acidic conditions, followed by amide coupling of the first encoding monomer. Because this set of building blocks comprised fewer than 16 compounds, a single monomer sufficed for encoding. At this point, each fraction was analyzed by LC-MS and subsequently pooled. The combined resin was subjected to Fmoc deprotection, and split into 18 parts. Orthogonal synthesis required encoding of the trifunctional building blocks prior to their installation. Accordingly, two encoding monomers were coupled, followed by a spacing Lys monomer which was Fmoc deprotected and Trt protected. Alloc deprotecting liberated the small molecule site of the conjugate, allowing for introduction of the trifunctional building blocks. The resin was pooled, subjected to Fmoc deprotection and split into 62 fractions. Each fraction was subjected to coupling of a carboxylic acid building block, followed by treatment with acyl chloride to ensure that all basic amines were efficiently capped. Elongation of the peptide tag was achieved through trityl deprotection and encoding with the corresponding monomers. The fractions were pooled and Fmoc deprotected. Coupling of trityl-protected valine as a spacing monomer set the stage for the Pd-mediated cross-coupling reactions. Accordingly, the library was divided into two parts to be used in the C—C and C—N coupling, respectively. Each library was split into 35 parts, and the fractions were functionalized with the respective Pd-mediated cross-coupling, trityl deprotected and coupled to the corresponding encoding monomers. The resin of each was pooled, Fmoc deprotected and coupled to the final spacing monomer which also encodes the library type: Lys and Arg for libraries resulting from C—C and C—N coupling, respectively. Standard TFA-mediated cleavage and solid phase extraction affords the final peptide-encoded small molecule libraries.

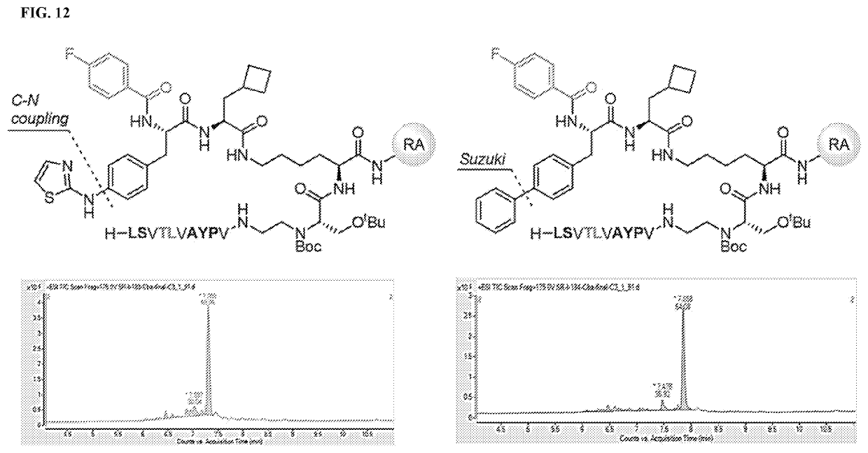

Each reaction used during the synthesis of the peptide-encoded small molecule libraries has been optimized and validated for each of the building blocks. Nonetheless, it was confirmed that these reactions provided the desired product even when performed sequentially as part of a 40-step synthesis. Two model substrates were prepared using the same sequence of procedures as during the combinatorial library synthesis (FIG. 12). Model substrates resulting from Pd-mediated C—C and C—N cross-coupling afforded product in good purity. Since combinatorial libraries can be neither analyzed nor purified, this synthetic efficiency is imperative and stands in contrast to examples from DELs were synthesis of single substrates under DNA-compatible conditions has been shown to afford mixtures of several products.

### De Novo Discovery of Small Molecule Ligands

Peptide-encoded libraries were synthesized according to the section “Synthesis of a Peptide-Encoded Small Molecule Library” to yield C—N and C—C cross-coupling-based libraries featuring 41 k and 39 k members, respectively. Affinity selections were performed as outlined in the section “Enrichment of Model Conjugates” using biotinylated human carbonic anhydrase IX (CA IX) as an immobilized target protein. nLC-MS/MS acquisition and subsequent sequencing were performed according to the section “MS/MS sequencing”. Ranking of identified hits according to enrichment analysis afforded several small molecules with selective enrichment for CA IX over a streptavidin control. The small molecules were readily synthesized on resin using the same reaction conditions as used during library synthesis. Alternatively, small molecules can be synthesized in solution. Moreover, the small molecules can be equipped with a functional group (e.g. biotin, fluorescent label, etc.) to allow validation of binding affinity or other desired properties. The small molecules identified from affinity selection of the C—N and C—C-based PELs were synthesized with pendant biotin and confirmed to be nanomolar binders for CA IX using BLI.

### INCORPORATION BY REFERENCE

All U.S. patents and U.S. and PCT patent application publications mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A conjugate having a structure represented by formula I or a salt thereof: 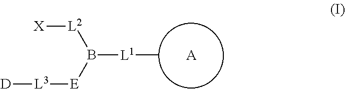
wherein
A is a resin;
B is a branching unit;
D is hydrogen, a carboxy protecting group, an amino protecting group, or an encoding peptide tag;
E is a cleavable moiety;
X is hydrogen, halo, amino, carboxy, protected, amino, protected carboxy, a small molecule, alkyl, alkene, alkyne, aryl, ketone, acyl, aldehyde, hydroxy, or a plurality of small molecules;
L¹ is a cleavable covalent linker;
L² is a covalent linker; and
L³ is a covalent linker.

2.-72. (canceled)

73. A method of identifying a ligand for a substrate comprising contacting the substrate with the conjugate of claim 1, wherein X is a small molecule or a plurality of small molecules; and D is a encoding peptide tag.

74. The method of claim 73, wherein the substrate is a protein.

75. The method of claim 73, wherein the substrate is a polynucleotide.

76. The method of claim 73, further comprising incubating the substrate with the conjugate.

77. The method of claim 73, further comprising cleaving a bond between L¹ and A.

78. The method of claim 73, further comprising cleaving a bond between B and L¹.

79. The method of claim 73, further comprising denaturing the substrate.

80. The method of claim 79, wherein the method further comprises a step of deconvoluting the ligand.

81. The method of claim 80, wherein the step of deconvoluting the ligand comprises:
cleaving L³, thereby yielding a free peptide tag;
optionally isolating the free peptide tag;
optionally purifying the free peptide tag; and
determining the molecular weight or retention time of the free peptide tag.

82. The method of claim 81, wherein L³ is cleaved via oxidative cleavage, acidic cleavage, or basic cleavage.

83. (canceled)

84. The method of claim 81, wherein the free peptide tag is isolated.

85. The method of claim 84, wherein free peptide tag is isolated by high performance liquid chromatography.

86. (canceled)

87. (canceled)

88. The method of claim 81, wherein the molecular weight of free peptide tag is determined.

89. The method of claim 88, wherein the molecular weight and the sequence of the free peptide tag is determined by mass spectrometry.

90. The method of claim 88, wherein the molecular weight and the sequence of the free peptide tag is determined by the analysis of mass fragmentation patterns.

91-93. (canceled)

94. The method of claim 81, wherein the sequence of the free peptide tag is determined.

95. The method of claim 94, wherein the sequence of the free peptide tag is determined by Edman degradation.

96. The method of claim 74, wherein identifying the ligand comprises comparing the molecular weight or retention time of the free peptide tag to a database or list comprising the identity of the small molecule or the plurality of small molecules.

97. A kit for performing the method of claim 74, wherein the kit comprises the conjugate as defined in claim 1, wherein D is hydrogen, amino, protected amino, carboxy, or protected carboxy and X is hydrogen, halo, amino, protected amino, or protected carboxy.

98.-128. (canceled)
